(19) United States
(12) Patent Application Publication
(10) Pub. No.: US 2023/0125563 A1
(43) Pub. Date: Apr. 27, 2023

(54) SYSTEM AND METHOD FOR REPAIRING A LIGAMENT

(71) Applicant: Pedro Juan LOREDO, Southlake, TX (US)

(72) Inventor: Pedro Juan LOREDO, Southlake, TX (US)

(21) Appl. No.: 17/509,410

(22) Filed: Oct. 25, 2021

(51) Int. Cl. A61F 2/08

(52) U.S. Cl. CPC A61F 2/0811; A61F 2/0805; A61F 2220/0075; A61F 2002/0852; A61F 2002/0888; A61F 2220/0016

(57) ABSTRACT

A surgical kit for repairing a damaged ligament includes a first anchor configured to be secured to a first bone during a surgical procedure to repair the damaged ligament. Suture tape is pre-attached to the first anchor. A second anchor is configured to secure a free end of the suture tape to a second bone adjacent the first bone during the surgical procedure.

### TECHNICAL FIELD

The present invention is directed to a system and method for repairing a ligament and, more particularly, to a system and method of repairing a ligament using anchors.

### BACKGROUND

The hand includes multiple ligaments that attach to the joints of the finger. Injury to these ligaments is commonly due to any hard force on the finger that causes the finger to be bent too far. This force can result in a partial tear or a complete tear of the ligament. A tear to the ligament results in instability of the joint, swelling, pain, bruising, and possible deformity of the finger. Repairing the ligament involves reattaching the torn ligament with internal sutures.

### SUMMARY

The present disclosure includes one or more of the features recited in the appended claims and/or the following features which, alone or in any combination, may comprise patentable subject matter.

According to a first aspect of the disclosed embodiments, a surgical kit for repairing a damaged ligament includes a first anchor configured to be secured to a first bone during a surgical procedure to repair the damaged ligament. Suture tape is pre-attached to the first anchor prior to the surgical procedure and extends from the first anchor to a free end. The suture tape is configured to extend over the damaged ligament. A second anchor is configured to secure the free end of the suture tape to a second bone adjacent the first bone during the surgical procedure.

In some embodiments of the first aspect, at least one suture may extend from the first anchor. The at least one suture may be configured to suture a first end of the damaged ligament to a second end of the damaged ligament during the surgical procedure. A suture needle may be secured to an end of the at least one suture. The at least one suture may include a pair of sutures extending from the first anchor.

Optionally, in the first aspect, at least one first anchor tine may extend from the first anchor. The first anchor tine may be configured to be inserted into the first bone to secure the first anchor to the first bone. At least one second anchor tine may extend from the second anchor. The second anchor tine may be configured to be inserted into the second bone to secure the second anchor to the second bone. Pliers may be provided to apply pressure to the first anchor to secure the first anchor to the first bone. The pliers may be configured to apply pressure to the second anchor to secure the second anchor to the second bone.

It may be desired, in the first aspect, that the second anchor may include an opening configured to receive the free end of the suture tape. An inner tine may extend into the opening of the second anchor. Pressure may be applied to the second anchor to collapse the opening of the second anchor onto the free end of the suture tape so that the inner tine secures to the free end of the suture tape.

According to a second aspect of the disclosed embodiments, a method for repairing a damaged ligament includes securing a first anchor to a first bone during a surgical procedure to repair the damaged ligament. The method also includes extending suture tape over the damaged ligament. The suture tape is pre-attached to the first anchor prior to the surgical procedure and extends from the first anchor to a free end. The method also includes securing the free end of the suture tape to a second bone adjacent the first bone during the surgical procedure with a second anchor.

In some embodiments of the second aspect, the method may also include suturing a first end of the damaged ligament to a second end of the damaged ligament with at least one suture extending from the first anchor during the surgical procedure. The method may also include suturing a first end of the damaged ligament to a second end of the damaged ligament with a needle secured to the at least one suture. The at least one suture may include a pair of sutures extending from the first anchor.

Optionally, in the second aspect, the method may also include inserting a first anchor tine extending from the first anchor into the first bone to secure the first anchor to the first bone. The method may also include inserting a second anchor tine extending from the second anchor into the second bone to secure the second anchor to the second bone. The method may also include applying pressure with pliers to the first anchor to secure the first anchor to the first bone. The method may also include applying pressure with the pliers to the second anchor to secure the second anchor to the second bone.

It may be desired, in the second aspect, that the method may also include positioning the free end of the suture tape in an opening of the second anchor. The method may also include applying pressure to the second anchor to collapse the opening of the second anchor onto the free end of the suture tape so that an inner tine extending into the opening secures to the free end of the suture tape.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a view of a surgical kit for repairing a damaged ligament including a side view of a primary anchor and a side view of a secondary anchor;

FIG. 2 is a front perspective view of the primary anchor used in the surgical kit to repair the damaged ligament;

FIG. 3 is a front elevation view of the secondary anchor used in the surgical kit to repair the damaged ligament;

FIG. 4 is a top view of a pair of pliers used to in the surgical kit to repair the damaged ligament;

FIG. 5 is a top view of a damaged ligament extending over a proximal bone and a distal bone;

FIG. 6 is a top view of the sutures of the primary anchor used to suture the damaged ligament shown in FIG. 5; and

FIG. 7 is a top view of the suture tape of the primary anchor extended across the damaged ligament and inserted into the secondary anchor.

### DETAILED DESCRIPTION

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

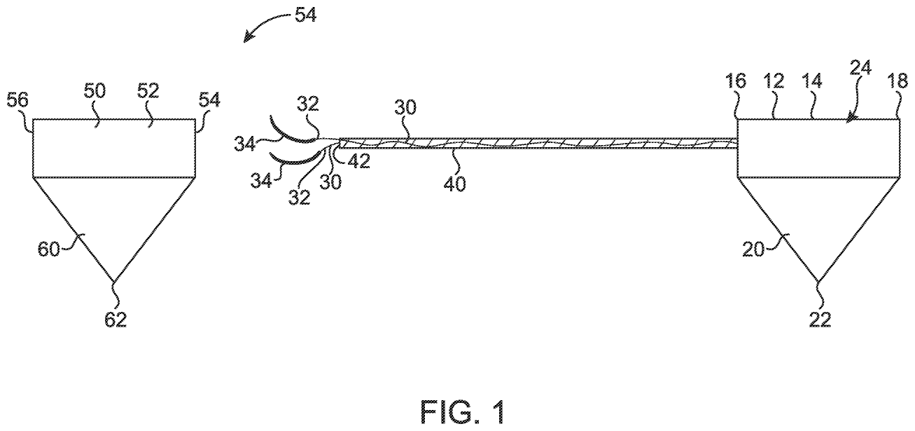

Referring now to FIG. 1, a surgical kit 10 for repairing a damaged ligament, for example a ligament in a patient's hand, includes a primary anchor 12. The primary anchor 12 incudes a generally rectangular base 14 that extends between a front end 16 and a back end 18. It will be appreciated that, in some embodiments, the primary anchor 12 may take any suitable shape, for example circular. A primary anchor tine 20 extends downward from base 14. In the illustrative embodiment, the primary anchor 12 includes a single primary anchor tine 20 that extends downward from the base 14 in a cone configuration to a point 22. In other embodiments, the primary anchor 12 may include any number of primary anchor tines 20 that extend to points 22. The primary anchor 12 also includes a top surface 24 that is configured to receive pressure from a pair of pliers 100, illustrated in FIG. 4, during a surgical procedure to repair the damaged ligament. The primary anchor tine 20 is configured to be pressed into a patient's bone by the pliers 100 during the surgical procedure so that the primary anchor 12 is secured to the patient's bone.

At least one suture 30 extends from the primary anchor 12. The at least one suture 30 extends from the base 14 to a free end 32. A needle 34 is secured to the free end 32. In the illustrative embodiment, the primary anchor 12 includes a pair of sutures 30 extending therefrom. It will be appreciated that the primary anchor 12 may include any number of sutures 30. In some embodiments, the at least one suture 30 is utilized to suture two ends of damaged ligament together during the surgical procedure to repair the damaged ligament. It will be appreciated that in some embodiments, the primary anchor 12 does not include the at least one suture 30, and the surgical procedure is performed without suturing the two ends of the damaged ligament together.

Suture tape 40 also extends from the primary anchor 12. The suture tape 40 extends from the base 14 to a free end 42. The suture tape 40 is configured to be positioned over the damaged ligament during the surgical procedure to repair the damaged ligament. The suture tape 40 is configured to increase a suture footprint allowing for increased soft tissue-to-bone contact in soft tissue repairs, e.g. ligament repairs. The suture tape 40 holds the ligament in place post-surgery to allow the ligament to reconnect to the bone. The suture tape 40 may be formed from an absorbable material that is absorbed into the patient's body over time post-surgery.

A secondary anchor 50 incudes a generally rectangular base 52 that extends between a front end 54 and a back end 56. It will be appreciated that, in some embodiments, the secondary anchor 50 may take any suitable shape, for example circular. A secondary anchor tine 60 extends downward from base 52. In the illustrative embodiment, the secondary anchor 50 includes a single secondary anchor tine 60 that extends downward from the base 52 in a cone configuration to a point 62. In other embodiments, the secondary anchor 50 may include any number of secondary anchor tines 60 that extend to points 62. The secondary anchor 50 also includes a top surface 64 that is configured to receive pressure from the pair of pliers 100 during the surgical procedure to repair the damaged ligament. The secondary anchor tine 60 is configured to be pressed into a patient's bone by the pliers 100 during the surgical procedure so that the secondary anchor 50 is secured to the patient's bone.

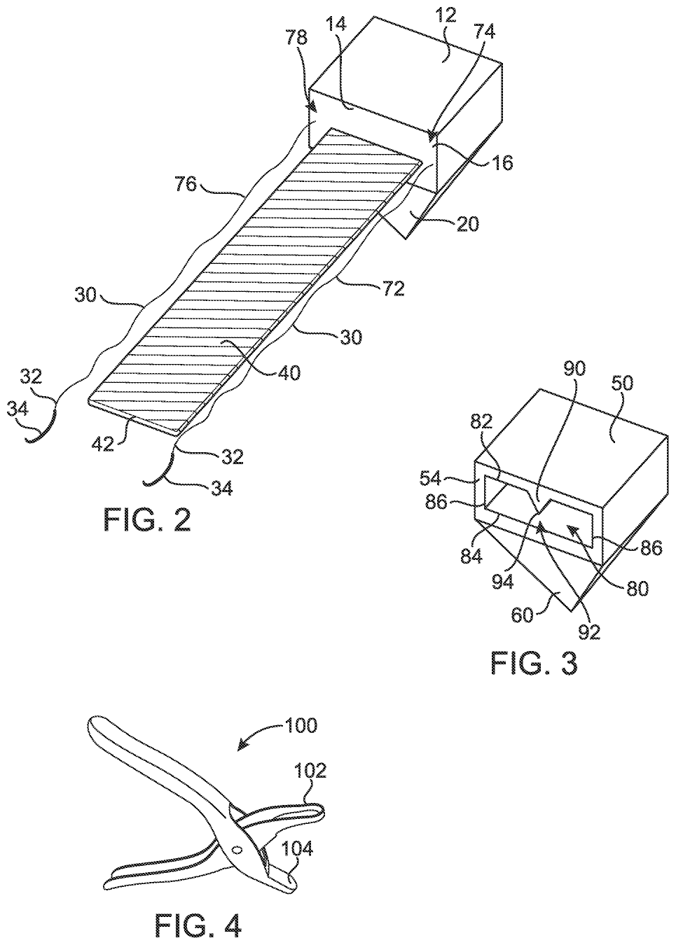

Referring to FIG. 2, in the exemplary embodiment, a pair of sutures 30 extend from the front end 16 of the base 14 of the primary anchor 12. Each of the pair of sutures 30 extends to a needle 34. The suture tape 40 also extends from the front end 16 of the base 14 of the primary anchor 12. The suture tape 40 is positioned between the pair of sutures 30. That is each of the sutures 30 is positioned on one of the sides of the suture tape 40. For example, a left suture 72 is positioned on a left side 74 of the suture tape 40, and a right suture 76 is positioned on a right side 78 of the suture tape 40. In other embodiments, the suture tape 40 and the sutures 30 may be oriented in different positions. For example, at least one suture 30 may be positioned below the suture tape 40.

Referring now to FIG. 3, the front end 54 of the secondary anchor 50 includes an opening 80 that is configured to receive the free end 42 of the suture tape 40 during the surgical procedure to repair the damaged ligament. The opening 80 is defined by a top wall 82, a bottom wall 84, and a pair of side walls 86. The secondary anchor 50 is positioned during the surgical procedure so that the front end 54 of the secondary anchor 50 faces the front end 16 of the primary anchor 12. Accordingly, the free end 42 of the suture tape 40 extends toward the secondary anchor 50. The free end 42 of the suture tape 40 is configured to be inserted into the opening 80.

An inner tine 90 extends downward from the top wall 82 toward the bottom wall 84. The inner tine 90 extends partially downward from the top wall 82 toward the bottom wall so that a slot 92 is formed between the inner tine 90 and the bottom wall 84. The inner tine 90 extends to a point 94. The slot 92 is formed between the point 94 and the bottom wall 84. The free end 42 of the suture tape 40 is configured to be inserted into the opening 80 so that the free end 42 of the suture tape 40 slides into the slot 92 under the point 94 of the inner tine 90. When pressure is applied to the secondary suture 50 to secure the secondary suture 50 to the bone, the inner tine 90 is pressed into the free end 42 of the suture tape 40 to secure the suture tape 40 in the secondary suture 50.

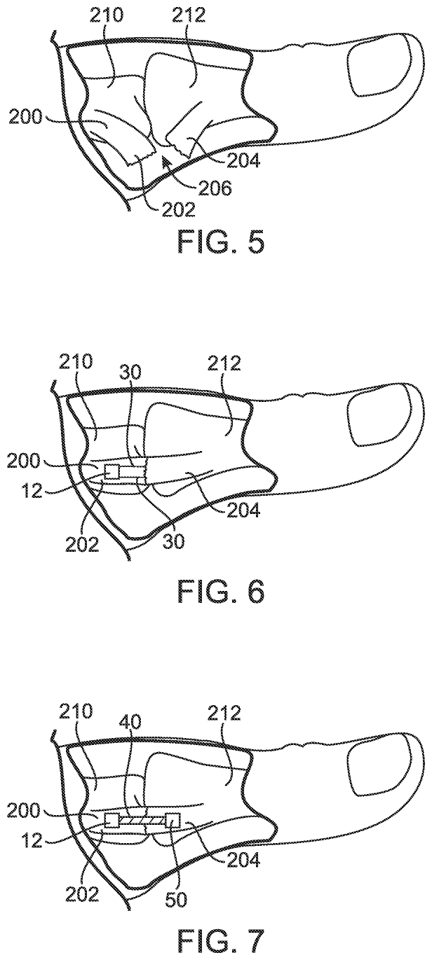

FIGS. 5-7 illustrate a method for repairing a damaged ligament 200, for example a hand ligament, having a proximal end 202 separated from a distal end 204. Referring now to FIG. 5, the proximal end 202 of the ligament 200 is secured to a proximal bone 210, and the distal end 204 of the ligament 200 is secured to a distal bone 212. A tear 206 is formed between the proximal end 202 and the distal end 204. In the exemplary embodiment, the method will be described with respect to securing the primary anchor 12 to the proximal bone 210 and proximal end 202 of the ligament 200, and securing the secondary anchor 50 to the distal bone 212 and the distal end 204 of the ligament 200; however, it will be appreciated that the primary anchor 12 may be secured to the distal bone 212 and the distal end 204 of the ligament 200, and the secondary anchor 50 may be secured to the proximal bone 210 and proximal end 202 of the ligament 200.

Referring to FIG. 6, the primary anchor 12 is positioned on the proximal bone 210 over the proximal end 202 of the damaged ligament 200. Using the pliers 100, a first arm 102 of the pliers 100 is positioned on the proximal bone 210, and an second arm 104 of the pliers 100 is positioned on the top surface 24 of the primary anchor 12. The arms 102, 104 of the pliers 100 are squeezed together to apply pressure to the primary anchor 12 so that the primary anchor tine 20 embeds within the proximal bone 210. The primary anchor 12 is secured in the proximal bone 210 so that the front end 16 faces the distal bone 212. Accordingly, the sutures 30 and the suture tape 40 extend toward the distal bone 212.

In one embodiments, the proximal end 202 and the distal end 204 of the ligament 200 are pulled together and sutured with the sutures 30.

That is, the needles 34 are utilized to thread the sutures 30 through the proximal end 202 and the distal end 204 of the ligament 200 to secure the proximal end 202 and the distal end 204 of the ligament 200 together. It will be appreciated that in some embodiments, the surgical kit 10 may not include the sutures 30 and the sutures 30 may not be used to secure the proximal end 202 and the distal end 204 of the ligament 200 together. For example, the ligament 200 may only have a partial tear and may not require suturing.

Referring now to FIG. 7, the secondary anchor 50 is positioned on the distal bone 212 over the distal end 204 of the damaged ligament 200. The secondary anchor 50 is positioned so that the front end 54 of the secondary anchor 50 faces the proximal bone 210. That is, the front end 54 of the secondary anchor 50 faces the primary anchor 12. Accordingly, the opening 80 of the secondary anchor 50 faces the primary anchor 12. The free end 42 of the suture tape 40 is inserted into the opening 80 through the slot 92. The suture tape 40 is pulled through the opening 80 until the suture tape 40 is secured against the damaged ligament 200.

Using the pliers 100, the first arm 102 of the pliers 100 is positioned on the distal bone 212, and the second arm 104 of the pliers 100 is positioned on the top surface 64 of the secondary anchor 50. The arms 102, 104 of the pliers 100 are squeezed together to apply pressure to the secondary anchor 50 so that the secondary anchor tine 60 embeds within the distal bone 212. Concurrently, the inner tine 90 embeds in the free end 42 of the suture tape 40 that has been inserted in the opening 80 to secure the suture tape 40 in the secondary anchor 50. In some embodiments, the top wall 82 collapses toward the bottom wall 84 to collapse the opening 80 so that the inner tine 90 embeds into the suture tape 40.

With the suture tape 40 secure across the damaged ligament 200, the surgical site is closed with the primary anchor 12 and the secondary anchor 50 positioned in the surgical site. In some embodiments, all of the elements of the primary anchor 12 and the secondary anchor 50 may be formed from an absorbable material that are absorbed into the patient's body over time post-surgery.

Any theory, mechanism of operation, proof, or finding stated herein is meant to further enhance understanding of principles of the present disclosure and is not intended to make the present disclosure in any way dependent upon such theory, mechanism of operation, illustrative embodiment, proof, or finding. It should be understood that while the use of the word preferable, preferably or preferred in the description above indicates that the feature so described can be more desirable, it nonetheless cannot be necessary and embodiments lacking the same can be contemplated as within the scope of the disclosure, that scope being defined by the claims that follow.

In reading the claims it is intended that when words such as “a,” “an,” “at least one,” “at least a portion” are used there is no intention to limit the claim to only one item unless specifically stated to the contrary in the claim. When the language “at least a portion” and/or “a portion” is used the item can include a portion and/or the entire item unless specifically stated to the contrary.

It should be understood that only selected embodiments have been shown and described and that all possible alternatives, modifications, aspects, combinations, principles, variations, and equivalents that come within the spirit of the disclosure as defined herein or by any of the following claims are desired to be protected. While embodiments of the disclosure have been illustrated and described in detail in the drawings and foregoing description, the same are to be considered as illustrative and not intended to be exhaustive or to limit the disclosure to the precise forms disclosed. Additional alternatives, modifications and variations can be apparent to those skilled in the art. Also, while multiple inventive aspects and principles can have been presented, they need not be utilized in combination, and many combinations of aspects and principles are possible in light of the various embodiments provided above.

1. A surgical kit for repairing a damaged ligament, the surgical kit comprising:
a first anchor configured to be secured to a first bone during a surgical procedure to repair the damaged ligament,
suture tape pre-attached to the first anchor prior to the surgical procedure and extending from the first anchor to a free end, the suture tape configured to extend over the damaged ligament, and
a second anchor configured to secure the free end of the suture tape to a second bone adjacent the first bone during the surgical procedure.

2. The surgical kit of claim 1, further comprising at least one suture extending from the first anchor, wherein the at least one suture is configured to suture a first end of the damaged ligament to a second end of the damaged ligament during the surgical procedure.

3. The surgical kit of claim 2, further comprising a suture needle secured to an end of the at least one suture.

4. The surgical kit of claim 2, wherein the at least one suture includes a pair of sutures extending from the first anchor.

5. The surgical kit of claim 1, further comprising at least one first anchor tine extending from the first anchor, wherein the first anchor tine is configured to be inserted into the first bone to secure the first anchor to the first bone.

6. The surgical kit of claim 1, further comprising at least one second anchor tine extending from the second anchor, wherein the second anchor tine is configured to be inserted into the second bone to secure the second anchor to the second bone.

7. The surgical kit of claim 1, further comprising pliers to apply pressure to the first anchor to secure the first anchor to the first bone.

8. The surgical kit of claim 7, wherein the pliers are configured to apply pressure to the second anchor to secure the second anchor to the second bone.

9. The surgical kit of claim 1, wherein the second anchor further comprises an opening configured to receive the free end of the suture tape.

10. The surgical kit of claim 9, further comprising an inner tine extending into the opening of the second anchor, wherein pressure is applied to the second anchor to collapse the opening of the second anchor onto the free end of the suture tape so that the inner tine secures to the free end of the suture tape.

11. A method for repairing a damaged ligament, the method comprising:
securing a first anchor to a first bone during a surgical procedure to repair the damaged ligament,
extending suture tape over the damaged ligament, wherein the suture tape is pre-attached to the first anchor prior to the surgical procedure and extends from the first anchor to a free end, and
securing the free end of the suture tape to a second bone adjacent the first bone during the surgical procedure with a second anchor.

12. The method of claim 11, further comprising suturing a first end of the damaged ligament to a second end of the damaged ligament with at least one suture extending from the first anchor during the surgical procedure.

13. The method of claim 12, further comprising suturing a first end of the damaged ligament to a second end of the damaged ligament with a needle secured to the at least one suture.

14. The method of claim 12, wherein the at least one suture includes a pair of sutures extending from the first anchor.

15. The method of claim 11, further comprising inserting a first anchor tine extending from the first anchor into the first bone to secure the first anchor to the first bone.

16. The method of claim 11, further comprising inserting a second anchor tine extending from the second anchor into the second bone to secure the second anchor to the second bone.

17. The method of claim 11, further comprising applying pressure with pliers to the first anchor to secure the first anchor to the first bone.

18. The method of claim 17, further comprising applying pressure with the pliers to the second anchor to secure the second anchor to the second bone.

19. The method of claim 11, further comprising positioning the free end of the suture tape in an opening of the second anchor.

20. The method of claim 19, further comprising applying pressure to the second anchor to collapse the opening of the second anchor onto the free end of the suture tape so that an inner tine extending into the opening secures to the free end of the suture tape.
